(19) United States
(12) Patent Application Publication
(10) Pub. No.: US 2022/0184292 A1
(43) Pub. Date: Jun. 16, 2022

(54) DUAL LUMEN DRAINAGE CANNULA WITH SINGLE OUTLET

(71) Applicant: CardiacAssist, Inc., Pittsburgh, PA (US)

(72) Inventors: Kelli Edwards, Glenshaw, PA (US); Patrick A. Kelly, North Huntingdon, PA (US); Michael J. Linehan, Pittsburgh, PA (US)

(73) Assignee: CardiacAssist, Inc., Pittsburgh, PA (US)

(21) Appl. No.: 17/686,122

(22) Filed: Mar. 3, 2022

Related U.S. Application Data
(63) Continuation of application No. PCT/US2020/052468, filed on Sep. 24, 2020.
(60) Provisional application No. 62/906,548, filed on Sep. 26, 2019.

(51) Int. Cl. A61M 1/36; A61M 60/113; A61M 60/38; A61M 60/859

(52) U.S. Cl. CPC A61M 1/3659; A61M 1/3667; A61M 60/859; A61M 60/38; A61M 60/113

(57) ABSTRACT

A dual lumen drainage cannula configured for use in a veno-arterial extracorporeal membrane oxygenation (VA ECMO) system includes a first drainage tube having a proximal end, a distal end, and at least one aperture defined in the distal end. The dual lumen drainage cannula further includes a second drainage tube having a proximal end, a distal end, and at least one aperture defined in the distal end. The dual lumen drainage cannula further includes an outlet fitting in fluid communication with the first drainage tube and the second drainage tube. The distal end of the second drainage tube is joined to a portion of the first drainage tube between the proximal and distal ends of the first drainage tube.

### CROSS REFERENCE TO RELATED APPLICATIONS

This application is a continuation of International Application No. PCT/US2020/052468, filed Sep. 24, 2020, which claims the benefit of priority to U.S. Provisional Patent Application Ser. No. 62/906,548, filed on Sep. 26, 2019, titled Dual Lumen Drainage Cannula With Single Outlet, the disclosure of which is incorporated herein by reference.

### BACKGROUND

### Field

The present disclosure generally relates to devices and methods for assisting a patient's heart with a cannula. More specifically, the present disclosure is related to cannula assemblies, systems including at least one cannula assembly, and methods of use thereof for medical procedures such as veno-arterial extracorporeal membrane oxygenation.

### Description of the Related Art

Veno-arterial extracorporeal membrane oxygenation (VA ECMO) is one method for treating right ventricular failure and respiratory failure percutaneously. A VA ECMO procedure draws blood from the right atrium and pumps it through an oxygenator and back into the arterial circulation via the femoral artery. VA ECMO bypasses the lungs and the heart completely, elevating arterial pressure and infusing blood into the arterial system with added oxygen and reduced carbon dioxide. One of the results of this therapy is that the blood that remains in the heart must be pumped by the heart to a higher pressure level in order to be ejected by the left ventricle because the VA ECMO system has elevated the arterial pressure to a higher level that represents a higher afterload to the pumping effort of the left ventricle.

In conventional VA ECMO systems, one drainage cannula is placed in the superior vena cava (SVC), the inferior vena cava (IVC), right atrium region by way of a femoral vein (typically) to drain blood therefrom and a separate, second return cannula is placed in an artery to return oxygenated (and cleansed from carbon dioxide) blood at a higher pressure. To drain additional blood from the pulmonary artery in conventional VA ECMO systems requires the insertion of a second drainage cannula (third total cannula) placed into the pulmonary artery by way of the jugular vein or other access site. Among the benefits of drawing blood from both the right atrium and the pulmonary artery is that the blood drained is fully mixed venous blood, including coronary circulation which drains into the right atrium, and that the right ventricle is unloaded to a greater extent. The use of multiple cannulas, however, consequently requires multiple cannula insertion sites, thereby increasing the risk of bleeding, vessel damage, and infection, as well as pain and discomfort to the patient.

While multi-lumen cannulas exist in the art, such cannulas may not be configured for draining blood flow from two separate sites. For example, a dual lumen cannula described in U.S. Pat. Nos. 9,168,352, 9,782,534, and 10,279,101, the disclosures of which are hereby incorporated by reference in their entireties, can be used to unload the right side of the heart by drawing blood from the right atrium through one lumen and infusing blood to the pulmonary artery through a second lumen.

A more recent innovation in VA ECMO systems utilizes a dual lumen cannula in which both lumens are used for drainage to the pump. Such a system is described in PCT Patent Application Publication No. WO 2016/054543, the disclosure of which is hereby incorporated by reference in its entirety. However, in the VA ECMO systems described in that document, the lumens of the cannula are separated by a “Y” connector into two outlets, which must be rejoined by a separate connector element to create a single lumen of flow into the pump. An example of one such prior art VA ECMO system is shown in FIG. 1, which illustrates a drainage cannula 1 inserted into the vasculature of a patient 6. The cannula 1 includes a first tube 2 of which an opening thereof is positioned in the pulmonary artery 3, and a second tube 4 of which an opening thereof is positioned in the right atrium 5 of the heart. The first tube 2 and the second tube 4 are fluidly separated from one another throughout the entirety of the cannula 1, and each of the first tube 2 and the second tube 4 drain blood toward separate outlets 7a, 7b of the cannula 1. Both outlets 7a, 7b of the cannula 1 are connected to a separate component, namely a Y-connector 8, which joins the flow from the two outlets 7a, 7b of the cannula 1 into a single flow path into a blood pump 9. In such VA ECMO systems, separation of the cannula 1 into two outlets 7a, 7b necessitates the use of the Y-connector 8. The separate outlets 7a, 7b allow for individual flow control through the first and second tubes 2, 4, for example by providing a valve at each outlet 7a, 7b. However, the necessitation of the Y-connector 8 to rejoin the flow of the separate outlets 7a, 7b may be disadvantageous in some circumstances.

### SUMMARY

In view of the foregoing, there exists a need for a dual lumen cannula, particularly for use in VA ECMO procedures, capable of draining blood from multiple vascular locations while having a single outlet lumen for supplying the drained blood to a pump. Embodiments of the present disclosure are generally directed to a VA ECMO system, a cannula assembly for a VA ECMO system, and a method of providing VA ECMO of a heart.

Embodiments of the present disclosure are directed to a veno-arterial extracorporeal membrane oxygenation (VA ECMO) system including a dual lumen drainage cannula. The dual lumen drainage cannula includes a first drainage tube having a proximal end, a distal end, and at least one aperture defined in the distal end; a second drainage tube having a proximal end, a distal end, and at least one aperture defined in the distal end; and an outlet fitting comprising a single lumen in fluid communication with the first drainage tube and the second drainage tube. The system further includes a blood pump having an inlet connected to the outlet fitting of the dual lumen drainage cannula, an oxygenator connected to an outlet of the blood pump, and an infusion cannula connected to an outlet of the oxygenator and configured for insertion into the vasculature of a patient. The distal end of the second drainage tube is joined to a portion of the first drainage tube between the proximal and distal ends of the first drainage tube.

In some embodiments, the infusion cannula is configured for insertion into a femoral artery of the patient.

In some embodiments, the at least one aperture of the first drainage tube is configured for draining blood from a pulmonary artery of the patient, and the at least one aperture of the second drainage tube is configured for draining blood from a right atrium of the patient.

In some embodiments, the first drainage tube extends coaxially relative to the second drainage tube.

In some embodiments, the proximal end of the first drainage tube is positioned distally of the outlet fitting.

In other embodiments, the present disclosure is directed to a dual lumen drainage cannula assembly configured for use in a veno-arterial extracorporeal membrane oxygenation (VA ECMO) system. The dual lumen drainage cannula includes a first drainage tube having a proximal end, a distal end, and at least one aperture defined in the distal end. The dual lumen drainage cannula further includes a second drainage tube having a proximal end, a distal end, and at least one aperture defined in the distal end. The dual lumen drainage cannula further includes an outlet fitting comprising a single lumen in fluid communication with the first drainage tube and the second drainage tube. The distal end of the second drainage tube is joined to a portion of the first drainage tube between the proximal and distal ends of the first drainage tube.

In some embodiments, the dual lumen drainage cannula further includes a retainer for indexing the proximal end of the first drainage tube within the proximal end of the second drainage tube.

In some embodiments, the at least one aperture of the first drainage tube is configured for draining blood from a pulmonary artery of a patient, and the at least one aperture of the second drainage tube is configured for draining blood from a right atrium of the patient.

In some embodiments, the first drainage tube extends coaxially relative to the second drainage tube.

In some embodiments, the proximal end of the first drainage tube is positioned distally of the outlet fitting.

Other embodiments of the present disclosure are directed to a method of providing veno-arterial extracorporeal membrane oxygenation (VA ECMO) of a heart. The method includes providing a dual lumen drainage cannula including a first drainage tube having a proximal end, a distal end, and at least one aperture defined in the distal end; a second drainage tube having a proximal end, a distal end, and at least one aperture defined in the distal end; and an outlet fitting comprising a single lumen in fluid communication with the first drainage tube and the second drainage tube. The distal end of the second drainage tube is joined to a portion of the first drainage tube between the proximal and distal ends of the first drainage tube. The method further includes inserting the dual lumen drainage cannula into a first site in a patient's vasculature, maneuvering the dual lumen drainage cannula through the patient's vasculature such that the distal end of the first drainage tube is at least within proximity of the patient's pulmonary artery and such that the distal end of the second drainage tube is at least within proximity of the patient's right atrium, draining blood through the first drainage tube and the second drainage tube to a blood pump, pumping drained blood through an oxygenator to reduce carbon dioxide content of the blood, and delivering oxygenated blood with reduced carbon dioxide content to a second site in the patient's vasculature.

In some embodiments, the outlet fitting of the dual lumen drainage cannula is connected to an inlet of the blood pump.

In some embodiments, the first drainage tube extends coaxially relative to the second drainage tube.

In some embodiments, the dual lumen drainage cannula further includes a retainer for indexing the proximal end of the first drainage tube within the proximal end of the second drainage tube.

In some embodiments, the proximal end of the first drainage tube is positioned distally of the outlet fitting.

Further details and advantages of the present disclosure will be understood from the following detailed description read in conjunction with the accompanying drawings.

### BRIEF DESCRIPTION OF THE DRAWINGS

FIG. 1 is a schematic view of VA ECMO system as known in the prior art;

FIG. 2 is a side view of a drainage cannula according to an embodiment of the present disclosure;

FIG. 3 is a side view of the drainage cannula of FIG. 2, with the second drainage tube removed for clarity;

FIG. 4A is a cross-sectional view of the drainage cannula taken alone line II-II of FIG. 2, according to an embodiment of the present disclosure;

FIG. 4B is a cross-sectional view of the drainage cannula taken alone line II-II of FIG. 2, according to another embodiment of the present disclosure;

FIG. 4C is a cross-sectional view of the drainage cannula taken alone line II-II of FIG. 2, according to a further embodiment of the present disclosure;

FIG. 5 is a cross-sectional view of detail A shown in FIG. 3, according to an embodiment of the present disclosure;

FIG. 6 is a cross-sectional view of the second drainage tube of the drainage cannula of FIG. 3;

FIG. 7 is a cross-sectional view of detail B in FIG. 6;

FIG. 8 is a cross-sectional view of the drainage cannula taken along line I-I of FIG. 2;

FIG. 9 is a cross-sectional view of detail C in FIG. 8, illustrating a transition portion at a distal end of a drainage cannula of the drainage cannula;

FIG. 10 is a schematic view of the drainage cannula of any of FIGS. 2-9 positioned in the superior vena cava of a body of a patient;

FIG. 11 is a schematic view of the drainage cannula of any of FIGS. 2-9 positioned inside a patient's heart;

FIG. 12 is a schematic view of a VA ECMO system, including the drainage cannula of any of FIGS. 2-9, according to an embodiment of the present disclosure; and

FIG. 13 is a cross-sectional view of the drainage cannula taken alone line III-III of FIG. 2, according to an embodiment of the present disclosure.

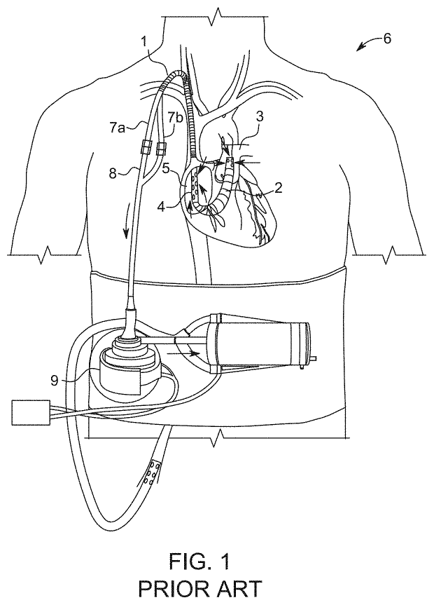

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments or aspects. Hence, specific dimensions and other physical characteristics related to the embodiments or aspects disclosed herein are not to be considered as limiting.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F. When used in relation to a cannula, catheter, or other device inserted into a patient, the term “proximal” refers to a portion of such device farther from the end of the device inserted into the patient. When used in relation to a cannula, catheter, or other device inserted into a patient, the term “distal” refers to a portion of such device nearer to the end of the device inserted into the patient.

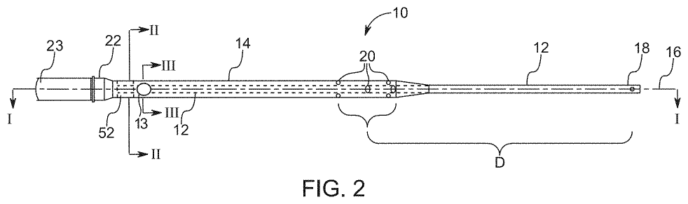

Referring to the drawings, in which like reference characters refer to like parts throughout the several views thereof, various embodiments of a dual lumen drainage cannula 10 (hereinafter referred to as “the drainage cannula 10”) are shown. With initial reference to FIG. 2, the assembled drainage cannula 10, according to one embodiment, generally includes a first drainage tube 12 having a first length and a second drainage tube 14 having a second length. The first length of the first drainage tube 12 is greater than the second length of the second drainage tube 14. The first drainage tube 12 is disposed partially within the second drainage tube 14, such that both the first drainage tube 12 and the second drainage tube 14 extend generally parallel to a central axis 16. In some embodiments, the first drainage tube 12 and the second drainage tube 14 may be arranged coaxially with one another about the central axis 16. An inner diameter of the second drainage tube 14 may be greater than an outer diameter of the first drainage tube 12 such that a flow cavity is formed inside the second drainage tube 14 around a portion of the first drainage tube 12 disposed within the second drainage tube 14. The first drainage tube 12 and the second drainage tube 14 are fluidly separated from one another along the entire length of the first drainage tube 12, such that a first fluid (e.g. blood drained from the pulmonary artery of a patient) carried through the first drainage tube 12 does not mix with a second fluid (e.g. blood drained from the right atrium of the patient) carried through the second drainage tube 14 until the first fluid reaches a proximal end of the first drainage tube 12.

One or both of the first drainage tube 12 and the second drainage tube 14 may be manufactured from a medical-grade material such as polyurethane. Alternatively, the tubes may be made from PVC or silicone, and may be dip molded, extruded, co-molded, or made using any other suitable manufacturing technique.

With continued reference to FIG. 2, a plurality of apertures 18 is provided at a distal end of the first drainage tube 12. The plurality of apertures 18 is desirably arranged in a circular pattern extending around a circumference of the first drainage tube 12. In some embodiments, the plurality of apertures 18 may be disposed in multiple groups provided at various sites on the first drainage tube 12. Similarly, a plurality of apertures 20 are provided at a distal end of the second drainage tube 14. The plurality of apertures 20 is desirably arranged in a circular pattern extending around the outer circumference of the second drainage tube 14. In alternative embodiments, the plurality of drainage apertures 20 may be arranged in groups disposed at various sites along the length of the second drainage tube 14. The apertures 18 of the first drainage tube 12 are separated along the length of the drainage cannula 10 from the apertures 20 of the second drainage tube 14 by a distance D. In some embodiments the distance D may be, or may correspond to, a vascular distance between the right atrium and the pulmonary artery of the patient such that the drainage cannula 10, when positioned in a patient for a VA ECMO procedure, may drain blood from the pulmonary artery via the apertures 18 of the first drainage tube 12 and drain blood from the right atrium via the apertures 20 of the second drainage tube 14. The distance D may vary based on the age and size of the patient, as well as the desired flow rates during the VA ECMO procedure. In some embodiments, the distance D may be, or may correspond to, a vascular distance between the right ventricle and the pulmonary artery of the patient such that the drainage cannula 10, when positioned in a patient for a VA ECMO procedure, may drain blood from the pulmonary artery via the apertures 18 of the first drainage tube 12 and drain blood from the right ventricle via the apertures 20 of the second drainage tube 14. In yet other embodiments, the apertures 20 may be positioned so as to drain blood from both the right atrium and the right ventricle simultaneously.

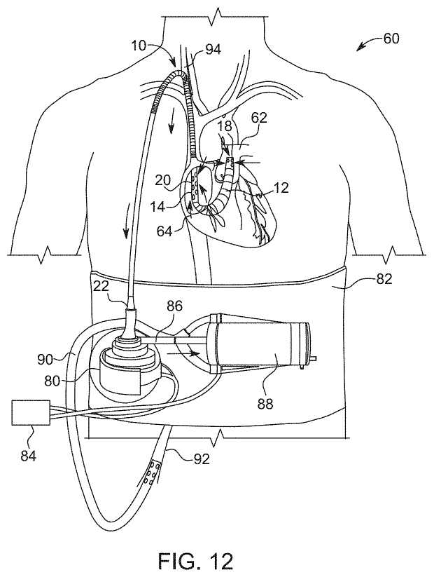

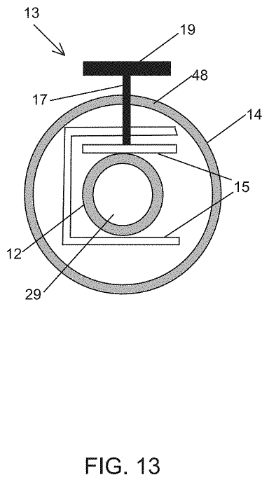

With continuing reference to FIG. 2, an outlet fitting 22 may be provided at the proximal end of the drainage cannula 10 for connecting the drainage cannula 10 to other medical devices, such as a blood pump 80 (see FIG. 12). The outlet fitting 22 is in fluid communication with the first drainage tube 12 and the second drainage tube 14 such that the drainage cannula 10 defines only a single outlet for draining fluid from both the first drainage tube 12 and the second drainage tube 14. In particular, the outlet fitting 22 defines a single outlet lumen 23 in fluid communication with the lumen of the first drainage tube 12 and the lumen of the second drainage tube 14 such that all flow in a proximal direction out of the drainage cannula 10 must flow through the single outlet lumen 23. The outlet fitting 22 may be, for example, a male hose barb, a luer connector, a male or female threaded connector, or a continuation of the second drainage tube 14 configured to fit over a hose barb.

With continuing reference to FIG. 2, in some embodiments a valve 13 may be provided on the first drainage tube 12 at a portion of the drainage cannula 10 that does not get inserted into the patient. For example, the valve 13 may be provided in proximity to the outlet fitting 22. The valve 13 allows a physician or other user to control fluid flow through the first drainage tube 12, and thereby to control the ratio of fluid drained from the first drainage tube 12 relative to fluid drained from the second drainage tube 14. Further details of the valve 13 will discussed herein in connection with FIG. 13.

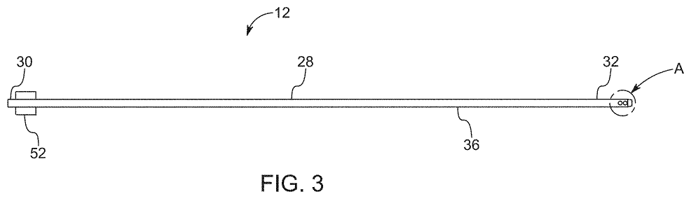

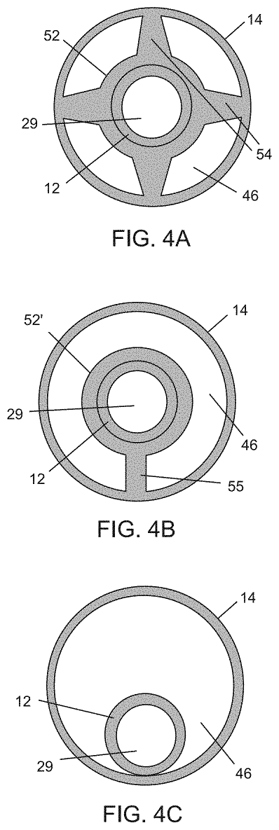

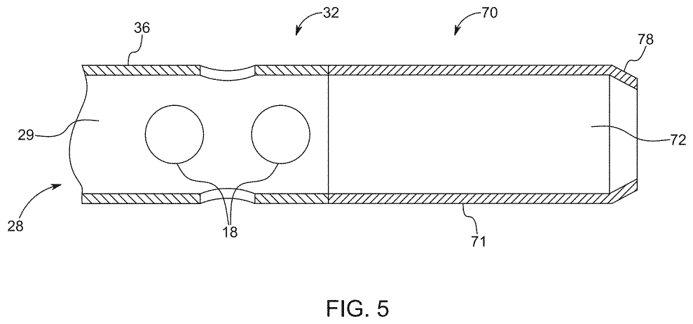

Referring now to FIG. 3, the first drainage tube 12 of the drainage cannula 10 is illustrated without the second drainage tube 14, which has been removed for clarity. The first drainage tube 12 has a first elongate body 28 having a working length of, for example, around 40-45 cm. The first elongate body 28 of the first drainage tube 12 is substantially cylindrical and extends from a first proximal end 30 to a first distal end 32. The first elongate body 28 defines a first lumen 29 (see FIGS. 4A-5) extending throughout the entire length of the first drainage tube 12. In some embodiments, a retainer 52 may extend radially from the first elongate body 28 to position the first drainage tube 12 within the second drainage tube 14. In some embodiments, the retainer 52 may position the first drainage tube 12 so as to be coaxial with the second drainage tube 14. In some embodiments, the retainer 52 may be located at or near the first proximal end 30 of the first elongate body 28. In some embodiments, a plurality of retainers 52 may be spaced along the length of the first elongate body 28. In still other embodiments, the retainer 52 may extend axially along a portion of the length first elongate body 28 or along the entire length of the first elongate body 28.

Various embodiments of the retainer 52 will now be described in greater detail with reference to FIGS. 4A-4C. Referring first to FIG. 4A, in some embodiments the retainer 52 may be star-shaped or cross-shaped and may include a plurality of straight or arcuate spokes or legs 54 extending radially between the first drainage tube 12 and the second drainage tube 14. Fluid in the second drainage tube 14 may flow through the portions of a second lumen 46 defined between the spokes or legs 54. Referring now to FIG. 4B, in other embodiments the retainer 52′ may include one or more ribs 55 extending radially between the first drainage tube 12 and the second drainage tube 14. In the embodiments of FIGS. 4A and 4B, the retainer 52, 52′ may be formed integrally with one or both of the first drainage tube 12 and the second drainage tube 14, or the retainer 52, 52′ may be a separate component joined to the first drainage tube 12 and/or the second drainage tube 14 during assembly of the drainage cannula 10. The spokes/legs 54 and/or the ribs 55 of the retainer 52, 52′ may index the first drainage tube 12 at a desired position within the lumen of the second drainage tube 14. For example, the spokes/legs 54 and/or the ribs 55 may index the first drainage tube 12 so as to be coaxial with the second drainage tube 14. Referring now to FIG. 4C, in some embodiments the retainer 52 may be omitted and the first drainage tube 12 may be directly connected or integrally formed with the second drainage tube 14. For example, the wall of the first drainage tube 12 may be in direct contact with the wall of the second drainage tube 14, such that the first drainage tube 12 is offset from the central longitudinal axis of the second drainage tube 14.

Referring now to FIG. 5, in some embodiments, the first distal end 32 of the first elongate body 28 may be attached to or integrally formed with a pliable distal tip 70 having a hollow structure defined by a tip sidewall 71. The distal tip 70 defines a tip lumen 72 in fluid communication with the first lumen 29 of the first elongate body 28 to allow fluid flow between the tip lumen 72 and the first lumen 29. A distal end of the distal tip 70 may include a first tapering section 78 that tapers radially inward in a distal direction for facilitating easier insertion of the first drainage tube 12 into the patient's body. The distal tip 70 may be made from a medical grade material such as polyurethane, or another material suitable for bonding to the material of the first elongate body 28. In some embodiments, the distal tip 70 may be manufactured from a material having a Shore durometer hardness of less than or equal to 85A to prevent injury and/or discomfort to the patient when the cannula 10 is placed into the patient. In some embodiments, the distal tip 70 may have a Shore durometer hardness of at least 5A less than the Shore durometer hardness of the first elongate body 28. Other embodiments of the distal tip 70 suitable for use with the drainage cannula 10 of the present disclosure are described in International Application No. PCT/US2019/036987, the disclosure of which is hereby incorporated by reference in its entirety.

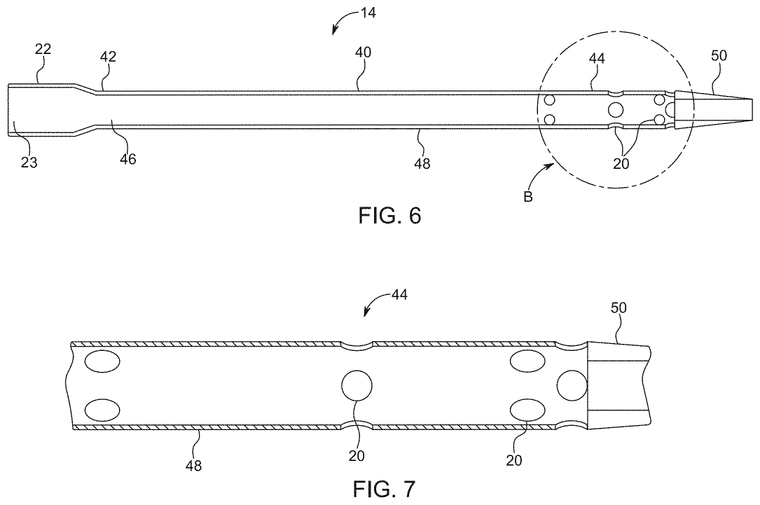

With reference to FIGS. 6-7, the second drainage tube 14 is illustrated separately from the other components of the drainage cannula 10 for clarity. The second drainage tube 14 has a second elongate body 40 having a working length of, for example, around 30 cm. The second elongate body 40 of the second drainage tube 14 may be substantially cylindrical and extends from a second proximal end 42 to a second distal end 44. The second elongate body 40 defines the second lumen 46 extending throughout the length of the second drainage tube 14. The second proximal end 42 may be connected to and/or integrally formed with the outlet fitting 22 (see FIG. 2). The second elongate body 40 of the second drainage tube 14 has a hollow structure defined by a second sidewall 48 extending circumferentially about the second elongate body 40. The second sidewall 48 has a substantially constant thickness throughout the length of the second elongate body 40, with a second tapering section 50 at the second distal end 44 of the second elongate body 40.The second tapering section 50 tapers distally to a sidewall thickness of substantially zero to define a smooth transition with the first sidewall 36 of the first drainage tube 12 (see FIG. 2) when the drainage cannula 10 is assembled.

With specific reference to FIG. 7, the second distal end 44 of the second drainage tube 14 is shown in detail. The plurality of apertures 20 is provided at the second distal end 44 of the second drainage tube 14. The plurality of apertures 20 may extend circumferentially around the second distal end 44. Each aperture 20 has a diameter of, for example, about 1.5 mm. The plurality of apertures 20 may be arranged in an alternating pattern of axially offset rows around the circumference of the second drainage tube 14. Each of the plurality of apertures 20 extends through the thickness of the second sidewall 48. The apertures 20 illustrated in FIGS. 6 and 7 may extend through the second sidewall 48 in a direction perpendicular to a longitudinal axis of the second elongate body 40. Alternatively, the plurality of apertures 20 may extend through the thickness of the second sidewall 48 in an angled manner with respect to the longitudinal axis of the second elongate body 40. For example, the plurality of apertures 20 may be arranged at an acute or obtuse angle with respect to a cross-sectional plane of the second drainage tube 14 extending perpendicular to the longitudinal axis of the second elongate body 40. In some embodiments, a wire mesh basket (not shown) is provided inside the second lumen 46 of the second drainage tube 14 at a location surrounding the apertures 20. The wire mesh basket supports and prevents the second sidewall 48 from collapsing due to being weakened by the formation of the plurality of apertures 20. In some embodiments, one or more sensors (not shown) may be provided at the second distal end 44 of the second drainage tube 14. The sensor(s) may be adapted for measuring, for example, local blood pressure and/or oxygen concentration.

The total cross-sectional area of the plurality of apertures 20 is desirably approximately equal to or greater than the cross-sectional area of the second lumen 46. If the cross-sectional area of the plurality of apertures 20 is less than the cross-sectional area of the second lumen 46, an undesirable pressure drop within the second drainage tube 14 may occur. This pressure drop reduces the flow throughput within the second lumen 46 and impairs the efficiency of the second drainage tube 14. Desirably, the total cross-sectional area of the plurality of apertures 20 exceeds the cross-sectional area of the second lumen 46 such that if one or more drainage apertures 20 becomes clogged, the total cross-sectional area of the remaining apertures 20 is equal to or greater than the cross-sectional area of the second lumen 46.

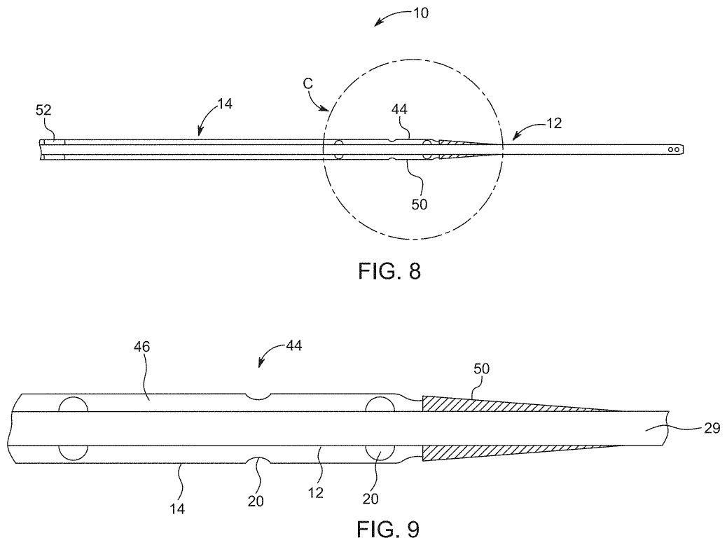

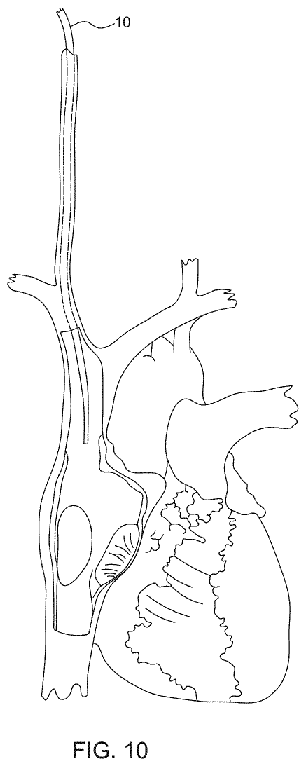

With reference now to FIGS. 8-9, the drainage cannula 10 shown in FIG. 2 is illustrated in cross section. The second distal end 44 of the second drainage tube 14 is fixedly attached to a mid-portion of the first drainage tube 12 along the length of a second tapering section 50, as shown in FIG. 9. For example, the second tapered section 50 may be in direct contact with the exterior surface of the first drainage tube 12 such that the circumferential inner surface of the second tapered section 50 of the second drainage tube 14 is secured to the circumferential outer surface of the first drainage tube 12. The first proximal end 30 of the first drainage tube 12 may be secured to the second drainage tube 14 at or near the second proximal end 42 by the retainer 52. In the embodiment shown, the first proximal end 30 of the first drainage tube 12 is located distally of the outlet fitting 22. In other embodiments, the first proximal end 30 of the first drainage tube 12 may extend into the outlet fitting 22.

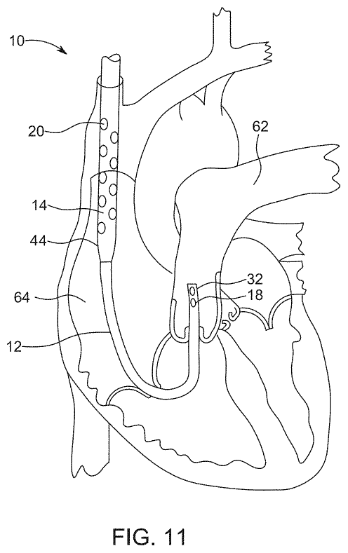

Having described several non-limiting embodiments of the drainage cannula 10 and the connector 22, an exemplary and non-limiting method for supporting the right heart of a patient using the drainage cannula 10 will now be described with reference to FIGS. 10-11. In use, the drainage cannula 10 is inserted into the pulmonary artery in a percutaneous procedure. Initially, a percutaneous entry needle (not shown) is used to access the patient's internal jugular vein (IJV). An introducer, such as a guidewire, is then inserted through the needle until the tip of the introducer is positioned in the upper portion of the inferior vena cava/right atrium (IVC/RA) junction. The needle can then be removed and a pulmonary wedge catheter inserted over the guidewire into the pulmonary artery. The introducer tip is then threaded into the pulmonary artery, and the wedge catheter is removed. The IJV is then serially dilated and the drainage cannula 10 is threaded along the introducer into the IJV, through the right ventricle, and into the pulmonary artery. The first distal end 32 of the first drainage tube 12 is sufficiently flexible about the central axis 16 so as to navigate the IJV, right ventricle, and pulmonary artery. The drainage cannula 10 may include insertion depth markers and radiopaque markers for aiding the user in placing the drainage cannula 10 in the right atrium. Once the position of the drainage cannula 10 is acceptable, the introducer may be removed and the drainage cannula 10 may clamped in place. For example, the drainage cannula 10 may be secured to the patient's neck using a suture. FIG. 11 shows the drainage cannula 10 positioned in the patient according to some embodiments of the disclosure. In particular, the second distal end 44 is positioned at least within proximity with the right atrium 64, while the first distal end 32 extends into the pulmonary artery 62.

Referring now to FIG. 12, the drainage cannula 10 is shown schematically as part of a VA ECMO system 60. As described above with reference to FIGS. 10 and 11, the first drainage tube 12 is positioned such that the plurality of apertures 18 thereof are located in the pulmonary artery 62, thereby allowing blood from the pulmonary artery 62 to drain through the plurality of apertures 18 and into the first lumen 29. The second drainage tube 14 is positioned such that the plurality of apertures 20 thereof are located in the right atrium 64, thereby allowing blood from the right atrium 64 to drain through the plurality of apertures 20 and into the second lumen 46. As noted above, in some embodiments, the plurality of apertures 20 may be located in the right ventricle in addition to or as an alternative of being positioned in the right atrium 64, thereby allowing blood from the right ventricle to drain through the plurality of apertures 20.

The outlet fitting 22 of the drainage cannula 10 may be connected to an inlet fitting of a blood pump 80. The pump 80 can be any centrifugal, axial, mixed, or roller pump that can produce adequate flowrates through the system. Several examples of pumps include, without limitation the TANDEMHEART pump manufactured by Cardiac Assist, Inc., the BIOMEDICUS pump manufactured by Medtronic, Inc., the ROTAFLOW pump manufactured by Jostra Medizintechnik AG, the CENTRIMAG pump manufactured by Levitronix, LLC, the SARNS DELPHIN pump manufactured by the Terumo Cardiovascular Group, the REVOLUTION pump manufactured by Cobe Cardiovascular, Inc, and others. The pump 80 can be secured to the patient, for instance with a holster 82 that holds the pump 80 with a strap or in a pocket. The holster 82 can be wrapped around the abdomen or shoulder or leg of the patient. A controller 84 may be provided for controlling the operation of the pump 80. The controller 84 may be built into the pump 80. The pump 80 further includes an outlet 86 for delivering blood to an oxygenator 88. The oxygenator 88 may be secured to the holster 82. The pump outlet 86 may be directly connected to the oxygenator 88, or the pump outlet 86 may be indirectly connected to the oxygenator 88 via a conduit or hose. The oxygenator 88 includes an oxygenation membrane or other element(s) for oxygenating blood flowing through the oxygenator 88. Oxygenated blood is delivered to an artery in the patient's body through an infusion cannula 90. While FIG. 12 illustrates the infusion cannula 90 connected to the patient's femoral artery 92, the infusion cannula in other embodiments may be connected to the patient's subclavian artery or another artery of the patient's vascular system.

In the VA ECMO system 60, the drainage cannula 10 allows the right atrial sourcing component, namely the second drainage tube 14, to drain the majority of venous flow, such as 4 liters per minute (lpm) out of a typical system flow of 5 lpm, leaving the pulmonary artery sourcing component, namely the first drainage tube 12, to drain the remaining 1 lpm.

Having described several non-limiting aspects of the drainage cannula 10 and the VA ECMO system 60, an exemplary and non-limiting method for bilateral unloading of a patient's heart using the drainage cannula 10 will now be described with continued reference to FIGS. 9-12.

In use, the drainage cannula 10 is inserted into the patient's vasculature in a percutaneous procedure prior to being connected to the other components of the VA ECMO system 60. Initially, a percutaneous entry needle (not shown) is used to access the patient's internal jugular vein 94 or the femoral vein. A guidewire, such as a guidewire having maximum diameter 0.038 in. (0.965 mm) and a minimum length of 170 cm, is inserted into the vasculature. In some aspects, the positioning of the guidewire is verified using an appropriate imaging technique. In the next step, the patient's active clotting time is checked for approximately 400 seconds.

The drainage cannula 10 may then be guided over the guidewire into the desired position within the patient's vasculature, shown in FIGS. 11 and 12. In some embodiments, an introducer (not shown) may be inserted into the first drainage tube 12 prior to guiding the drainage cannula 10 over the guidewire. In some aspects, the drainage cannula 10 may be guided into a desired position using indicia, such as a radiopaque marker located in the first distal end 32 of the first drainage tube 12, that is visualized under fluoroscopy, transthoracic echocardiography, or cineangiography. The position of the drainage cannula 10 may be guided and verified by an imaging system described in WO 2015/139031, the disclosure of which is hereby incorporated by reference in its entirety. After noting and recording the location of the drainage cannula 10, the introducer (if utilized) can be removed.

To connect the drainage cannula 10 to the blood pump 80, a wet-to-wet, or other type, of a connection is made between the drainage cannula 10 and the pump 80. After verifying the correct positioning and insertion depth of the drainage cannula 10, the drainage cannula 10 can be secured to the patient, such as by suturing with a suture wing. The patient's active clotting time is checked for approximately 180-220 seconds before turning on the blood pump 80 to circulate the patient's blood through the VA ECMO system 60. During use, fluid drained from the pulmonary artery 62 via the first drainage tube 10 and fluid drained from the right atrium 64 via the second drainage tube 64 flow proximally out of the drainage cannula 10 via the outlet fitting 22 and into the blood pump 80. The blood pump 80 pumps the blood received from the drainage cannula 10 to the oxygenator 88 to oxygenate the blood, which is then returned to the patient via the infusion line 90. After use, the pump 80 may be turned off and the pump inlet and outlet may be clamped. Any sutures securing the drainage cannula 10 to the patient may be removed, and the drainage cannula 10 removed from the patient. The puncture site may then be treated and dressed. Additional details of a VA ECMO procedure are described in PCT Application Publication No. WO 2016/054543, the disclosure of which is hereby incorporated by reference in its entirety.

Referring now to FIG. 13, in some embodiments, the valve 13 may be provided on the first drainage tube 12 as discussed herein with reference to FIG. 2. In some embodiments, the valve 13 may be a pinch valve including clamping jaws 15, a shaft 17, and a knob 19. The jaws 15 may be positioned on substantially opposite outer surfaces of the first drainage tube 12. A physician or other user may rotate the knob 19 to turn the shaft 17, thereby spreading the jaws 15 apart or drawing the jaws 15 together, as desired. Drawing the jaws 15 together may pinch the first drainage tube 12 to reduce the cross-sectional area of the first lumen 29, thereby limiting fluid flow through the first drainage tube 12. In some embodiments, the jaws 15 may be positioned inside the second drainage tube 14, and the shaft 17 may extend through a sealed hole in the sidewall 48 of the second drainage tube 14 to connect to the knob 19. While the valve 13 is shown and described herein as a pinch valve, it should be understood that any suitable type of valve such as a ball valve, a gate valve, or a butterfly valve may be utilized.

While several embodiments of a drainage cannula are shown in the accompanying figures and described hereinabove in detail, other embodiments will be apparent to, and readily made by, those skilled in the art without departing from the scope and spirit of the invention. For example, it is to be understood that this disclosure contemplates, to the extent possible, that one or more features of any embodiment can be combined with one or more features of any other embodiment. Accordingly, the foregoing description is intended to be illustrative rather than restrictive.

1. A veno-arterial extracorporeal membrane oxygenation (VA ECMO) system comprising:
a dual lumen drainage cannula comprising:
a first drainage tube having a proximal end, a distal end, and at least one aperture defined near the distal end;
a second drainage tube having a proximal end, a distal end, and at least one aperture defined in near the distal end; and
an outlet fitting comprising a single lumen in fluid communication with the first drainage tube and the second drainage tube,
a blood pump having an inlet connected to the outlet fitting of the dual lumen drainage cannula;
an oxygenator connected to an outlet of the blood pump; and
an infusion cannula connected to an outlet of the oxygenator and configured for insertion into the vasculature of a patient,
wherein the distal end of the second drainage tube is secured to a portion of the first drainage tube between the proximal and distal ends of the first drainage tube.

2. (canceled)

3. The VA ECMO system according to claim 1, wherein the at least one aperture of the first drainage tube is spaced apart from the at least one aperture of the second drainage tube by a distance such that the at least one aperture of the first drainage tube is positionable within a pulmonary artery of the patient to drain blood from the pulmonary artery while the at least one aperture of the second drainage tube is positionable within a right atrium of the patient to drain blood from the right atrium of the patient.

4. The VA ECMO system according to claim 1, wherein the first drainage tube extends coaxially relative to the second drainage tube.

5. The VA ECMO system according to claim 1, wherein the proximal end of the first drainage tube is positioned distally of the outlet fitting.

6. A dual lumen drainage cannula configured for use in a veno-arterial extracorporeal membrane oxygenation (VA ECMO) system, the dual lumen drainage cannula comprising:
a first drainage tube having a proximal end, a distal end, and at least one aperture defined near the distal end;
a second drainage tube having a proximal end, a distal end, and at least one aperture defined near the distal end; and
an outlet fitting comprising a single lumen in fluid communication with the first drainage tube and the second drainage tube,
wherein the distal end of the second drainage tube is secured to a portion of the first drainage tube between the proximal and distal ends of the first drainage tube.

7. The dual lumen drainage cannula according to claim 6, further comprising a retainer for indexing the first drainage tube within the second drainage tube.

8. The dual lumen drainage cannula according to claim 6, wherein the at least one aperture of the first drainage tube is spaced apart from the at least one aperture of the second drainage tube by a distance such that the at least one aperture of the first drainage tube is positionable within a pulmonary artery of the patient to drain blood from the pulmonary artery while the at least one aperture of the second drainage tube is positionable within a right atrium of the patient to drain blood from the right atrium of the patient.

9. The dual lumen drainage cannula according to claim 6, wherein the first drainage tube extends coaxially relative to the second drainage tube.

10. The dual lumen drainage cannula according to claim 6, wherein the proximal end of the first drainage tube is positioned distally of the outlet fitting.

11. A method of providing veno-arterial extracorporeal membrane oxygenation (VA ECMO) of a heart, the method comprising:
providing a dual lumen drainage cannula comprising:
a first drainage tube having a proximal end, a distal end, and at least one aperture defined near the distal end;
a second drainage tube having a proximal end, a distal end, and at least one aperture defined near the distal end; and
an outlet fitting comprising a single lumen in fluid communication with the first drainage tube and the second drainage tube,
wherein the distal end of the second drainage tube is secured to a portion of the first drainage tube between the proximal and distal ends of the first drainage tube;
inserting the dual lumen drainage cannula into a first site in a patient's vasculature;
maneuvering the dual lumen drainage cannula through the patient's vasculature such that the distal end of the first drainage tube is at least within proximity of the patient's pulmonary artery and such that the distal end of the second drainage tube is at least within proximity of the patient's right atrium;
draining blood through the first drainage tube and the second drainage tube to a blood pump;
pumping drained blood through an oxygenator to reduce carbon dioxide content of the blood; and
delivering oxygenated blood with reduced carbon dioxide content to a second site in the patient's vasculature.

12. The method according to claim 11, wherein the outlet fitting of the dual lumen drainage cannula is connected to an inlet of the blood pump.

13. The method according to claim 11, wherein the first drainage tube extends coaxially relative to the second drainage tube.

14. The method according to claim 11, wherein the dual lumen drainage cannula further comprises a retainer for indexing the proximal end of the first drainage tube within the proximal end of the second drainage tube.

15. The method according to claim 11, wherein the proximal end of the first drainage tube is positioned distally of the outlet fitting.

16. The VA ECMO system according to claim 1, further comprising a valve configured to selectively control a ratio of fluid drained through the first drainage tube relative to fluid drained through the second drainage tube.

17. The VA ECMO system according to claim 1, further comprising a retainer extending between the first drainage tube and the second drainage tube to index the first drainage tube within the second drainage tube.

18. The VA ECMO system according to claim 17, wherein the retainer includes one or more spokes extending radially between the first drainage tube and the second drainage tube.

19. The VA ECMO system according to claim 18, wherein the one or more spokes index the first drainage tube so as to be coaxial with the second drainage tube.

20. The dual lumen drainage cannula according to claim 6, further comprising a valve configured to selectively control a ratio of fluid drained through the first drainage tube relative to fluid drained through the second drainage tube.

21. The dual lumen drainage cannula according to claim 7, wherein the retainer includes one or more spokes extending radially between the first drainage tube and the second drainage tube.
